(19) United States
(12) Patent Application Publication
(10) Pub. No.: US 2025/0339153 A1
(43) Pub. Date: Nov. 6, 2025

(54) SYSTEM METHOD AND DEVICE FOR OCCLUDING AN ATRIAL APPENDAGE

(71) Applicant: Module Med LLC, Sugar Land, TX (US)

(72) Inventors: Kevin Zhenmei Liao, Sugar Land, TX (US); Kenneth Kangxiong Liao, Bellaire, TX (US)

(73) Assignee: Module Med LLC, Sugar Land, TX (US)

(21) Appl. No.: 19/197,730

(22) Filed: May 2, 2025

Related U.S. Application Data
(60) Provisional application No. 63/642,651, filed on May 4, 2024.

(51) Int. Cl. A61B 17/12; A61B 17/00

(52) U.S. Cl. CPC A61B 17/12122; A61B 17/12031; A61B 17/12159; A61B 2017/00477; A61B 2017/00862; A61B 2017/1205

(57) ABSTRACT

A system, method, and device for occluding an anatomical cavity, such as an atrial appendage are provided. The system includes a biocompatible plug assembly comprising a patch and strip made of, for example, bovine pericardium, where the strip is flexible and foldable on itself to reduce its vertical profile, as well as a surgical occlusion device configured to deliver the strip into an anatomical cavity to pack the anatomical cavity. The patch may be secured to an opening of the anatomical cavity, to create a secure plug preventing fluid or material passage. Applied to a left atrial appendage, the system and method reduce the risk of cardioembolic strokes from atrial fibrillation.

### CROSS-REFERENCE TO RELATED APPLICATIONS

Pursuant to 35 U.S.C. § 119(e), this application claims the benefit of U.S. Provisional Application No. 63/642,651, filed on May 4, 2024, which is incorporated herein by reference in its entirety.

### GOVERNMENT CONTRACT

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to devices for reducing reliance on blood thinners to reduce the risk of stroke and, more particularly, to devices and systems for surgical occlusion of an atrial appendage in cardiac surgery.

### BACKGROUND

Atrial fibrillation (AF) is the most common clinically important cardiac arrhythmia, occurring in approximately 0.4% to 1% of the general population and increasing with age to more than 8% in those over 80 years old. In the presence of AF, the upper chambers of the heart pump in an irregular way, out of sync with the lower chambers, and there is a decreased flow of blood inside the left atrium of the heart. Because of the reduced blood flow, there is a particular risk of blood clots forming within the left atrial appendage (LAA), a finger-like extension or sac projecting from the main body of the lef atrium. If the clots dislodge from the LAA and travel through the arteries in the heart, they can cause a stroke.

For patients with atrial fibrillation who face an elevated risk of stroke, doctors often recommend the use of a blood thinner in order to prevent or reduce the risk of clot formation within the LAA. If patients are unable to take a blood thinner because of risk of bleeding or falls, doctors may recommend a procedure to occlude LAA percutaneously or surgically.

Currently one method of closing LAA is conventional surgical closure during concomitant open heart surgery, especially mitral valve surgery. Conventional surgical closure has a high failure rate because the LAA cavity can recanalize, primarily due to high tension along the suture line and the LAA cavity remaining filled with fluid.

Another way of closing LAA is through catheter-inserted closure devices such as those sold under the trade name Watchman™ and LARIAT™. Indeed, several devices to achieve LAA occlusion have been proposed. U.S. Pat. No. 10,660,649 to Ad teaches a adjustably inflatable cuff positionable by catheter into an atrial appendage. U.S. Patent Pub. No. 20200138448 by Dasnurkar et al. and U.S. Pat. No. 9,566,073 to Kassab et al. similarly teach a catheter-delivered inflatable balloon to occlude an atrial appendage.

In addition, some have proposed surgically applying an external LAA occlusion device, such as those sold under the trade name AtriClip™. U.S. Pat. No. 10,925,615 to Deville et al. likewise teaches a recapturable clip and device for externally occluding fluid passageway of a hollow tissue structure such as an atrial appendage.

Still, despite increasing evidence demonstrating the beneficial reduction of stroke after occluding LAA during open heart surgery—especially during mitral valve surgery—surgeons do not frequently perform concomitant LAA occlusion due, at least in part, to lacking an internal LAA occluder device that can be easily inserted surgically.

Thus, although various proposals have been made to occlude an atrial appendage, none of those in existence combine the characteristics of the present invention. Therefore, there remains a need for an atrial appendage occluder that can be deployed during open heart surgery.

### SUMMARY

The present disclosure is directed to a surgical occluder and related systems and methods for closing or sealing an anatomical opening or cavity to prevent fluid or material passage.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In accordance with one embodiment, the surgical occluder is configured to facilitate the installation of a biocompatible strip into a target anatomical area, such as a cavity defined by an atrial appendage, and a patch sized and configured to block any opening to or orifice of such target area. For the sake of brevity, an atrial appendage, and in particular, the left atrial appendage (LAA), may be one such target anatomical area referenced throughout this disclosure. However, it is to be understood that any number of openings can be occluded by the system, method, and device herein described without departing from the invention.

To that end, the surgical occluder may comprise a base securable to a biocompatible plug assembly, a guide rod removably securable to the base, and a delivery mechanism configured to install or deposit the plug into the target anatomical area.

In some embodiments, the base may comprise a bottom having an inside surface, an outside surface, an outer edge, and a sidewall itself having a top edge and a bottom edge, the sidewall extending upward from a central portion of the inside surface of the bottom. The outside of the bottom of the base is configured to removably secure to a biocompatible patch and strip, which define the plug assembly, such that the patch and strip may be maintained in position for deployment and occlusion of an anatomical opening or cavity. Indeed, in some embodiments, the bottom edge of the base sidewall and edge of the bottom of the base itself may define notches to facilitate placement and retention of sutures for securing the plug assembly to the outside of the bottom of the base.

The biocompatible patch and strip may be formed of biocompatible bovine pericardium, without limitation. That is, other biocompatible materials may be used without departing from the invention. In an embodiment, the biocompatible strip may be formed as a long, straight strip, or created by cutting a spiral or serpentine pattern from a rectangular segment to produce an elongated, flexible strip capable of being folded or coiled to reduce its vertical profile. Alternatively, the strip may be folded into a helical, cylindrical, or spherical shape. This may be particularly desirable in instances where horizontal expansion in the anatomical opening or cavity is desired. One end of the folded strip may be secured to one side of the biocompatible patch, such as a bottom side of a bovine pericardium patch, by a suture passed through both components to secure them to one another.

The patch may be sized and configured to cover an anatomical opening or cavity, such as the LAA. As such, it is contemplated that the particular size and shape of the patch, and indeed, width and length of the strip, may vary depending on an individual patient's anatomical shape and size, or as otherwise needed or desired. The patch may also be secured with the strip prior to use or attached intraoperatively.

The surgical occluder may further comprise a guide rod having a proximal end and a distal end, in which the proximal end is securely yet removably couplable to the inside of the sidewall. In some embodiments, means for securing the guide rod to the base sidewall may be provided, although it is contemplated that the guide rod may be sized for a secure friction fit or configured for a snap fit within the sidewall. Still, in some embodiments, means for securing the guide rod to the base sidewall may comprise, for example only and not limitation, a detent disposed near the proximal end of the guide rod securely received through a hole defined by the sidewall. The detent may be defined by a tapered protrusion to permit forcible withdrawal from the sidewall hole, effectuating temporary securement of the guide rod in position while allowing intentional release by a user. In some embodiments, the detent may comprise a spring-biased element configured to expand outward once passed through the sidewall hole, thereby retaining the guide rod in position until manually compressed or deformed to permit release. In still other embodiments, the guide rod may be releasably securable to the sidewall by a screw system or even by tying or suturing the components together in manner that will be known to those of ordinary skill in the art.

The delivery mechanism of the surgical occluder may take a variety of forms operative to deposit the plug assembly, and in particular, the strip, into the anatomical cavity. In some exemplary embodiments, the surgical occluder may comprise a shaft defining a hollow interior, an exterior surface, a first open end, and a second open end, the second open end fitted with a shell selectively closeable around the base and plug assembly.

The selectively closeable shell is configured to transition between an open configuration that permits insertion and removal of the guide rod into the hollow shaft, and a closed configuration that retains the biocompatible patch and strip compactly against the outside surface of the base.

In some embodiments, the shell comprises a plurality of leaves hingedly secured to the shaft and collectively forming a hollow interior in the closed configuration, with each leaf optionally defining a hole to receive a suture for securing the shell closed.

A method of occluding an atrial appendage or other anatomical opening or cavity may therefore comprise: securing a first end of the biocompatible strip to a central portion of the bottom side of the biocompatible patch to define a plug; folding the biocompatible strip toward the patch; removably securing a top side of the patch to the outside of the bottom of the base; slidably inserting a distal end of the guide rod into the hollow interior of the shaft from the second open end of the shaft; closing the shell around the biocompatible patch and strip. During a surgical procedure, such as open heart surgery, then, the method may further comprise inserting a terminal end of the shell of the plug delivery mechanism into a target anatomical site of a patient; removing the plug delivery mechanism from the guide rod at the first end of the shaft such that the folded strip remains within the target anatomical site; removing the guide rod from the sidewall of the base; suturing the patch to an orifice defining an opening to the target anatomical site; and removing the base from the patch.

It is contemplated that providing a surgical occluder, system and method for using the same, according to the disclosure and claims provided below may aid the safe construction and installation of a biocompatible plug assembly to internally occlude a target anatomical area, in particular, the LAA.

Thus, it is an object of the invention to enable atrial appendage occlusion during open heart surgery.

It is another object of the invention to reduce the risk of stroke by occluding the LAA and preventing the release of LAA-originating blood clots into systemic circulation.

It is another object of the invention to effectuate atrial appendage occlusion during open heart surgery with biocompatible materials.

It is yet another object of the invention to provide a biocompatible implant material as a plug assembly which supports endothelialization after implantation.

It is still another object of the invention to reduce the risk of thrombosis or inflammation by promoting natural tissue integration through endothelial cell coverage of the implanted surface.

One or more of the above-disclosed embodiments, in addition to certain alternatives, are provided in further detail below with reference to the attached figures. The disclosed subject matter is not, however, limited to any particular embodiment disclosed.

### BRIEF DESCRIPTION OF THE DRAWINGS

FIG. 1 shows an exemplary biocompatible patch for use with a surgical occluder in accordance with one embodiment of the invention.

FIG. 2 shows an exemplary biocompatible strip for use with a surgical occluder in accordance with one embodiment of the invention.

FIG. 3 shows an alternative view of the exemplary biocompatible patch of FIG. 1 and exemplary biocompatible strip of FIG. 2.

FIG. 4 shows another view of the exemplary embodiment of the biocompatible patch and strip of FIG. 3.

FIGS. 5A and 5B illustrate alternative views of one exemplary method of securing the biocompatible strip to the biocompatible patch in a compressed manner in accordance with an embodiment of the invention.

FIG. 6A shows an exemplary base of a surgical occluder in accordance with one embodiment of the invention.

FIG. 6B shows the exemplary base of FIG. 6A engaged with a biocompatible patch and strip in accordance with one embodiment of the invention.

FIG. 7A shows an exemplary guide rod of a surgical occluder in accordance with one embodiment of the invention.

FIG. 7B shows the exemplary guide rod of FIG. 7A engaged with the exemplary base of FIG. 6A in accordance with one embodiment of the invention.

FIGS. 8A-B illustrate successive views of an exemplary shell and shaft of the surgical occluder engaged with the exemplary guide rod, base, and biocompatible patch and strip of FIG. 7B in accordance with one embodiment of the invention.

FIG. 8C shows an another exemplary view of a shell and shaft of the surgical occluder in accordance with one embodiment of the invention.

FIGS. 9A shows exemplary positioning of a surgical occluder with a partial view of a left atrial appendage as a step of inserting a biocompatible patch and strip into such appendage in accordance with one embodiment of the method for occluding an atrial appendage.

FIGS. 9B-C show the steps of inserting a biocompatible patch and strip into an atrial appendage with a surgical occluder in accordance with one embodiment of the method for occluding an atrial appendage.

FIG. 10 illustrates removal of the exemplary guide rod of the surgical occluder from the exemplary base of the surgical occluder in accordance with one embodiment of the method for occluding an atrial appendage.

FIGS. 11 and 12 illustrate removal of the exemplary base of the surgical occluder in accordance with one embodiment of the method for occluding an atrial appendage.

FIGS. 13 and 14 illustrate removal of exemplary stitches in accordance with one embodiment of the method for occluding an atrial appendage.

The disclosed embodiments may be better understood by referring to the figures in the attached drawings, as provided below. The attached figures are provided as non-limiting examples for providing an enabling description of the method and system claimed. Attention is called to the fact, however, that the appended drawings illustrate only typical embodiments of this invention and are therefore not to be considered as limiting of its scope. One skilled in the art will understand that the invention may be practiced without some of the details included in order to provide a thorough enabling description of such embodiments. Well-known structures and functions have not been shown or described in detail to avoid unnecessarily obscuring the description of the embodiments.

### DETAILED DESCRIPTION

Having summarized various aspects of the present disclosure, reference will now be made in detail to that which is illustrated in the drawings. While the disclosure will be described in connection with these drawings, there is no intent to limit it to the embodiment or embodiments disclosed herein. Rather, the intent is to cover all alternatives, modifications and equivalents included within the spirit and scope of the disclosure as defined by the appended claims.

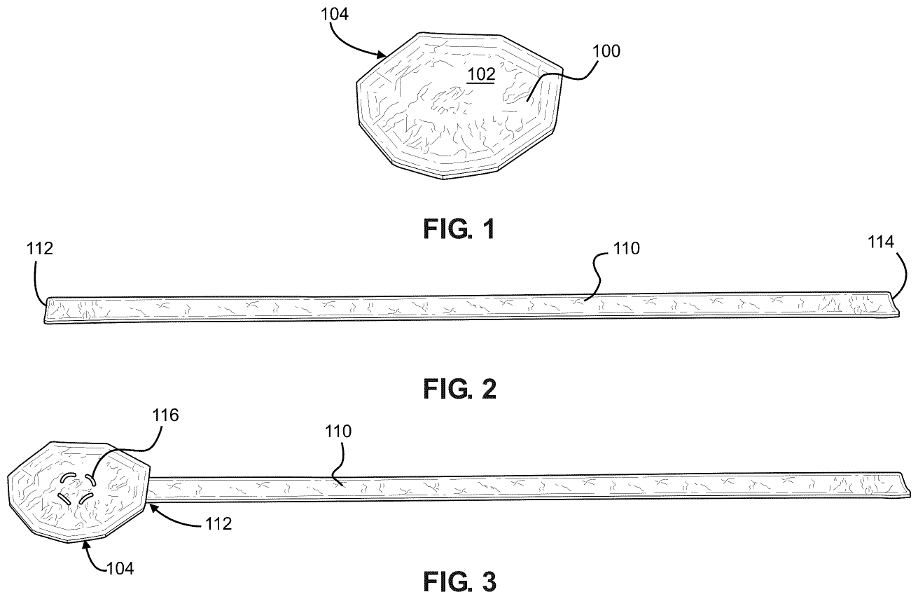

With reference to FIGS. 1-3 a biocompatible patch 100 having a top side 102 and a bottom side 104 may be sized and configured to cover an anatomical opening or cavity, such as a human LAA. As such, it is contemplated that the particular size and shape of the patch 100, and indeed, width and length of the strip, may vary depending on an individual patient's anatomical shape and size, or as otherwise needed or desired.

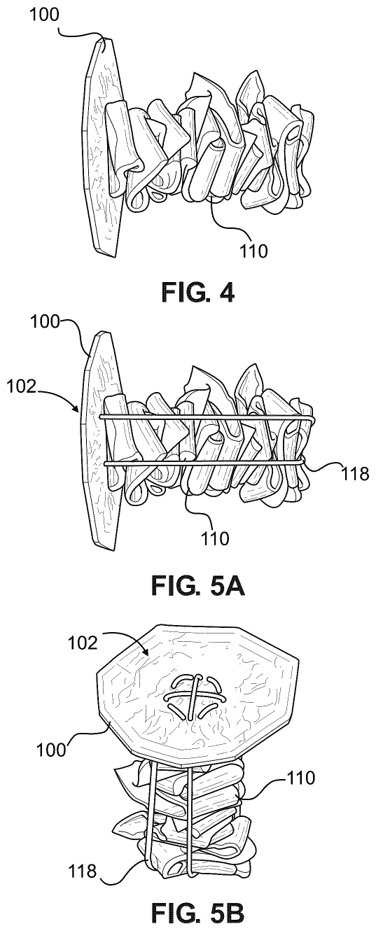

An exemplary biocompatible strip 110 may be formed as a long, straight strip, having a first end 112 and a second end 114. However, in some embodiments, the strip may be created by cutting a spiral or serpentine pattern from a material segment to produce an elongated, flexible strip capable of being folded or coiled to reduce its vertical profile, as in FIG. 4. Returning to FIG. 3, the first end 112 of the strip 110 may be secured to a central portion of the bottom side 104 of the patch 100, by one or more sutures 116 passed through both components to secure them to one another to define a plug assembly. The biocompatible strip 110 may be secured to the patch 100 prior to use or attached intraoperatively.

The biocompatible patch and strip may be formed of a number of materials without limitation. For example only, the patch 100 and strip 110 may comprise biocompatible bovine pericardium. Bovine pericardium is a membrane that surrounds the heart of a cow. It is typically treated with glutaraldehyde before it is used in human. Bovine pericardium is a biomaterial that has been widely used in human cardiovascular systems such as heart valves, atrium and vascular wall repair/replacement. It has thus far been demonstrated safe, effective and biocompatible after many years of in vivo use. After implantation, the surface of the material can become covered by the body's own endothelial cells, forming a natural tissue lining. This process, known as endothelialization, helps the implant integrate more effectively with surrounding tissue and reduces the risk of complications such as blood clots or inflammation.

Turning to FIGS. 5A-B, it is contemplated that means for compressing and maintaining the strip 110 in a folded position during application may be desired. Therefore, in some embodiments, a suture 118 may be disposed around the folded strip 110 and through to be accessible through the top 102 of the patch 100, such that the suture 118 can be released or cut following surgical application to allow the strip 110 to unfurl or expand and fill the LAA or other anatomical cavity and effectuate occlusion.

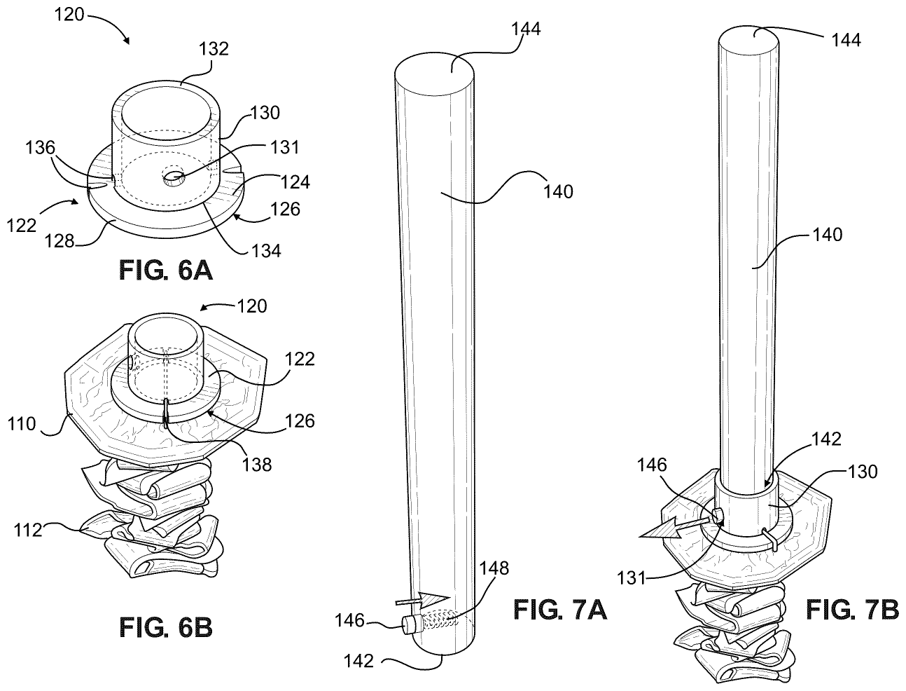

With reference to FIG. 6A, a surgical occluder base 120 may comprise a bottom 122 having an inside surface 124, an outside surface 126, an outer edge 128, and a sidewall 130 extending upward from a central portion of the inside surface 124 of the bottom 122. The outside surface 126 of the bottom 122 of the base 120 may be configured to removably secure to the biocompatible patch 100 and strip 110 plug assembly, such that the patch 110 and strip 112 may be maintained in position for deployment and occlusion of an anatomical opening or cavity. Indeed, in some embodiments, a bottom edge 132 of the base sidewall 130 and outer edge 128 of the bottom 122 of the base 120 itself may define a plurality of notches 136 to facilitate placement and retention of sutures 138, shown in FIG. 6B, for securing the patch 100 and strip 110 to the outside surface 126 of the bottom 122 of the base 120.

Turning to FIGS. 7A-B, a surgical occluder guide rod 140 may be defined by a proximal end 142 and a distal end 144. The proximal end 142 may be configured to securely yet removably couple with the inside of the sidewall 130. In some embodiments, means for securing the guide rod to the base sidewall may be provided. Means for securing the guide rod 140 to the base sidewall 130 may comprise, for example only and not limitation, a detent 146 disposed near the proximal end 142 of the guide rod 140 securely received through a hole 131 defined by the sidewall 130 and more clearly visible in FIG. 6A. Returning to FIGS. 7A-B, The detent 146 may be defined by a tapered protrusion to permit forcible withdrawal from the sidewall hole 131, effectuating temporary securement of the guide rod in position while allowing intentional release by a user. In some embodiments, as in the exemplary embodiment shown in the figures, the detent 146 may comprise a spring-biased element 148 configured to expand outward once passed through the sidewall 130 hole 131, thereby retaining the guide rod 140 in position until manually compressed or deformed to permit release.

Still, it is contemplated that the guide rod 140 may be sized for a secure friction fit with or configured for a secure snap fit within the sidewall 130. Indeed, in some embodiments, the guide rod 140 may be releasably securable to the sidewall 130 by a screw system or even by tying or suturing the components together in manner that will be known to those of ordinary skill in the art. As such, the foregoing is offered by way of example only, and not limitation.

Likewise, the material comprising the guide rod 140 will not limit the invention. For instance, the guide rod may comprise carbon fiber, braided wire, stainless steel, titanium, polymer composites, high-density polyethylene or polypropylene and other materials known to those of skill in the art which may be selected for characteristics comprising, for instance, strength, biocompatibility, weight, flexibility, etc.

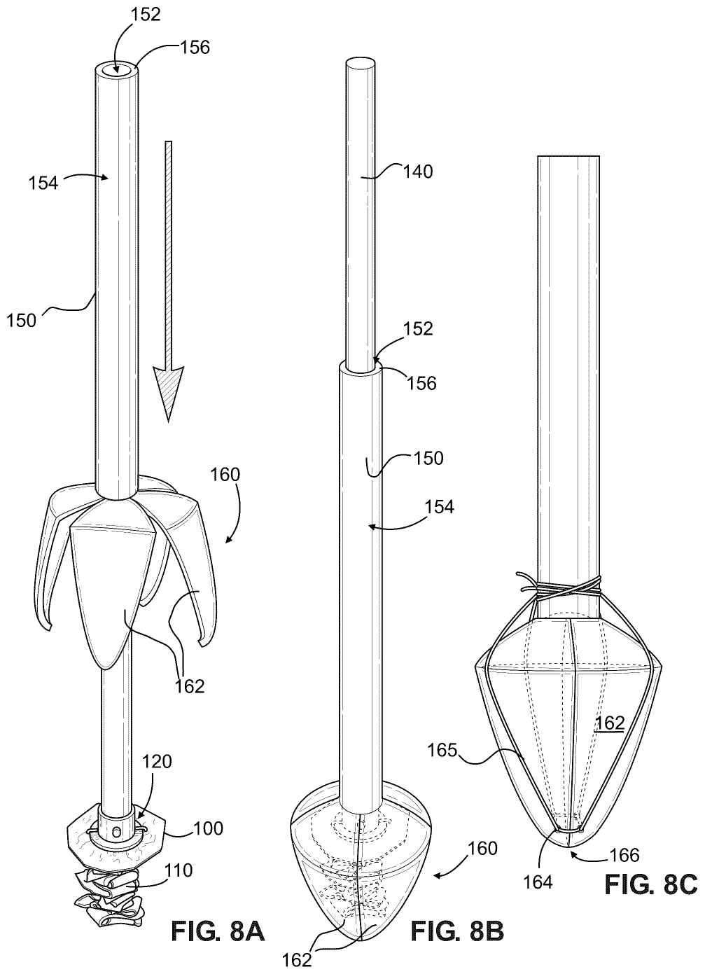

With reference to FIGS. 8A-B, one exemplary embodiment of a delivery mechanism of the surgical occluder may comprise a shaft 150 defining a hollow interior 152 configured to slidably receive the guide rod 140 therein, an exterior surface 154, a first open end 156, and a second open end (obscured), the second open end fitted with a shell 160 selectively closeable around the base 120 and patch 100 and strip 110 plug assembly.

The selectively closeable shell 160 is configured to transition between an open configuration, shown in FIG. 8A, that permits insertion and removal of the guide rod 140 into the hollow shaft 150, and a closed configuration, shown in FIG. 8B, that retains the biocompatible patch and strip compactly against the outside surface of the base (obscured within the shell 150).

In some embodiments, the shell comprises a plurality of leaves 162 hingedly secured to the shaft 150 and collectively forming a hollow interior in the closed configuration. In some embodiments, the leaves of the selectively closeable shell may be formed from a flexible or resilient biocompatible material, such as silicone, polyurethane, or a medical-grade thermoplastic, to allow controlled deformation during deployment. The leaves 162 may be biased toward either of the open or closed configuration, depending on the desired mechanical behavior of the shell 160 during insertion and placement of the plug assembly. For example, the leaves 162 may be spring-biased to open when not constrained, or alternatively, may be naturally relaxed in the closed position and spread open by insertion of the guide rod. The leaves 162 may be hingedly attached to the shaft 150 in any manner known to those of ordinary skill in the art in order to permit rotation or bending of each leaf 162 relative to the shaft 150, facilitating articulation and controlled enclosure of the patch 100 and strip 110. For example only, and not limitation, the leaves may be hingedly attached to the exterior surface of the shaft by flexible living hinges integral to the leaf material, mechanical hinges, or even by pivot pins or rivets passing through aligned holes in the leaves and shaft.

As such, it will be understood that the shaft 150 and leaves 162 may be formed from a unitary piece of biocompatible material capable of providing a flexible hinge at the junction between the shaft 150 and leaves 162. Suitable materials may include, without limitation, silicone, polypropylene, polyethylene, polyetheretherketone (PEEK), or other thermoplastics exhibiting sufficient flexibility at thin cross-sections while maintaining rigidity in thicker regions. Still, in some embodiments, the shaft 150 may be formed from a rigid biocompatible material, such as stainless steel or titanium, with the leaves 162 attached by separate hinges or connected by flexible polymeric strips operating as living hinges.

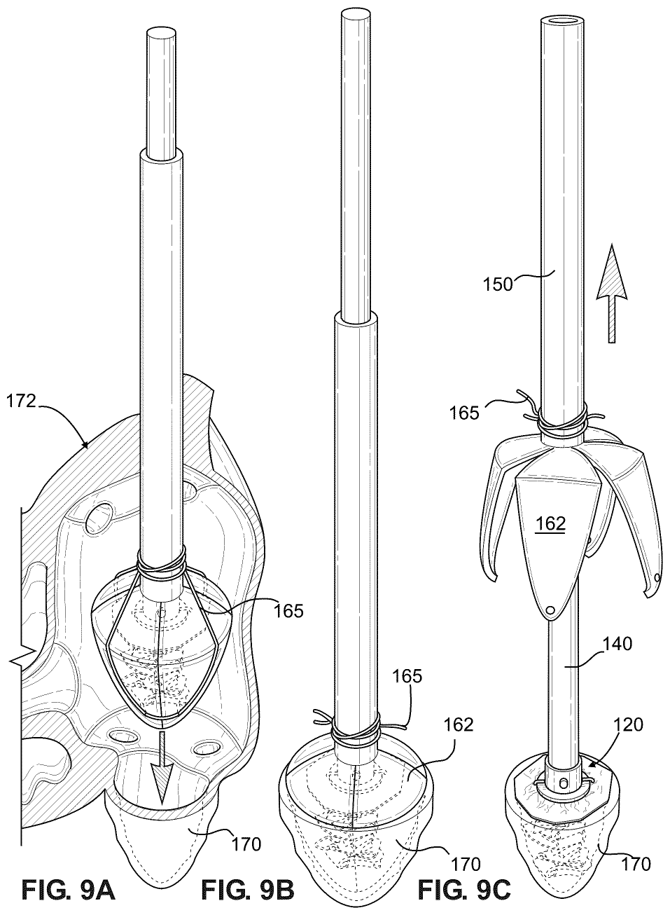

With reference to FIG. 8C, each leaf 162 may also define one or more holes 164 formed near a distal end 166, the holes 164 configured to receive a suture 165 or other tensioning element, enabling the leaves 162 to be drawn together and secured in the closed configuration until released by cutting, untying or the like. These mechanical features may allow the shell to conform to anatomical variations during use and improve retention of the patch assembly in situ. For instance, FIGS. 9A-C, illustrate exemplary insertion of the shell into an atrial appendage 170 extending from a patient's heart 172. Note that the exemplary appendage has been visually isolated from the remaining anatomical structure in FIGS. 9B-C simply for clarity. This is not meant to illustrate extraction of any appendages or cavities for occlusion, but merely to reduce visual clutter. Indeed, it is contemplated that a user will insert the surgical occluder and plug assembly into a patient's body over the course of surgery, such as open-heart surgery.

In any event, cutting the suture 165 that holds the leaves 162 in the closed position allows the leaves 162 to move to the open position, thereby enabling slidable removal of the shaft 150 from the guide rod 140, base 120, and patch assembly.

In some embodiments, the distal end 166 of the leaves 162 may be curved or blunt in order to avoid and/or prevent injury to the anatomical cavity during use.

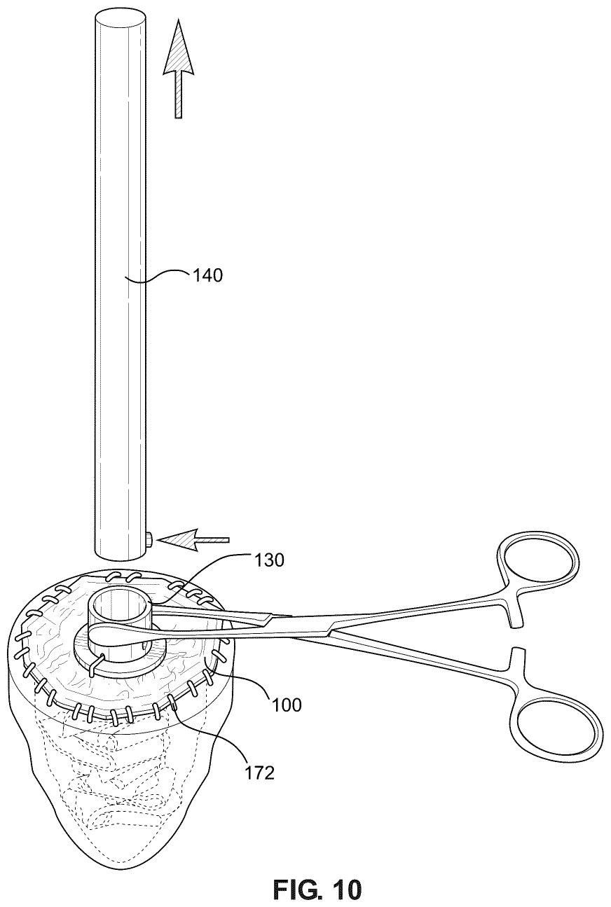

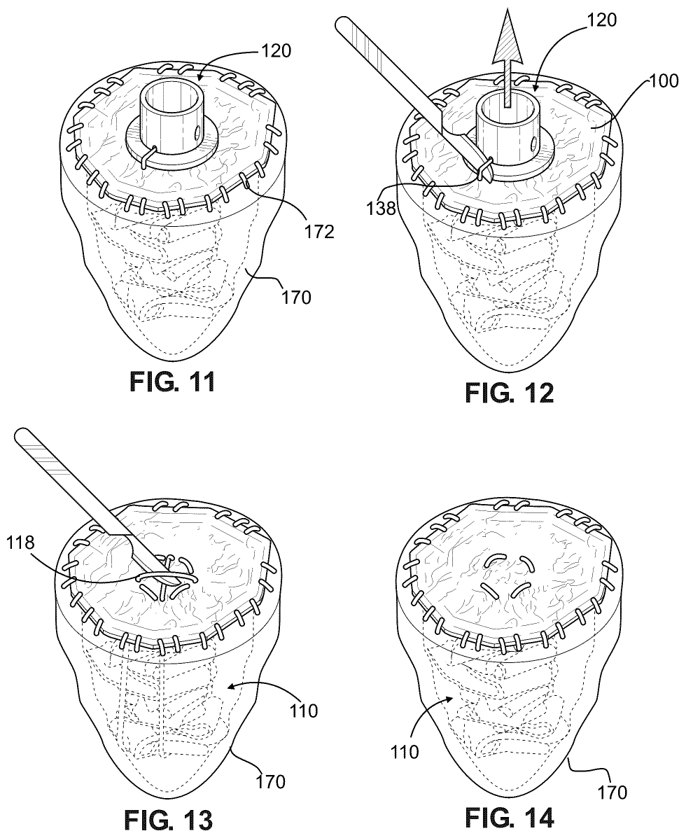

Turning to FIG. 10, installation of the plug assembly may further comprise removing the guide rod 140 from the base sidewall 130 and securing the patch 100 to an orifice (obscured) defining an opening to the target anatomical site, here the exemplary atrial appendage 170, with sutures 172. FIG. 11 is shown, therefore, with the rod removed from the base 120 and the patch secured to the atrial appendage 170 with the sutures 172. Thus, it may be seen that during surgery, the folded strip may be used to pack an anatomical cavity, here the atrial appendage 170 and the patch 110, sutured to the cavity orifice, forms a seal around such orifice to secure the strip in place within the cavity.

Continuing with the method, as in FIG. 12, the base 120 may be removed from the patch 110 by cutting suture 138. Then, in FIGS. 13 and 14 the folded strip 110 may be allowed to decompress within the cavity of the atrial appendage 170, or other anatomical target, in order to more fully occupy the space, by cutting sutures 118.

It will be understood that sutures deployed with the system and throughout performance of the method may comprise any biocompatible materials as needed or desired to perform such function. In some cases, the sutures may comprise non-absorbable materials such as polypropylene or polyester to ensure secure attachment between, for example only and not limitation, the base and the patch, while allowing flexibility to accommodate movement or deformation of the patch during placement. However, it is also contemplated that removal of any deployed stitches may be impractical or impossible. Therefore, it is contemplated that the sutures compressing the folded strip and securing the patch to the anatomical target, for example, may comprise, without limitation, biocompatible, absorbable materials such as polydioxanone (PDS), polyglycolic acid (PGA), or polyglactin 910 (Vicryl), selected to provide temporary securement of the patch during delivery and early healing, with gradual resorption to minimize long-term foreign material within the body.

In some embodiments, a metal brush may be used to brush the internal surface of LAA before pericardium plug is inserted into the cavity in order to promote adhesion of LAA cavity and biocompatible strip.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### CONCLUSIONS, RAMIFICATIONS, AND SCOPE

While certain embodiments of the invention have been illustrated and described, various modifications are contemplated and can be made without departing from the spirit and scope of the invention. For example, the shape, size, and configuration of the base, sidewall, patch, strip, guide rod, delivery mechanism, or shell may be varied; different biocompatible materials may be used for the patch and strip; and alternative mechanisms may be employed to secure or release the patch and strip; and the device may be adapted for use in different anatomical sites or occluding various types of openings or cavities. Accordingly, it is intended that the invention not be limited, except as by the appended claim(s).

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the system, method, and device for occluding an atrial appendage with which that terminology is associated. In general, the terms used in the following claims should not be constructed to limit the system, method, and device for occluding an atrial appendage to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the system, method, and device for occluding an atrial appendage is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the system, method, and device for occluding an atrial appendage.

What is claimed is:

1. A system for occluding an anatomical cavity during a surgery, comprising:
a biocompatible patch having a top side and a bottom side, the patch being sized and configured to cover the anatomical cavity, and
a biocompatible strip defining a flexible, elongated body foldable upon itself to reduce its vertical profile, the strip having a first end and a second end, the first end configured to be secured to a central portion of the bottom side of the patch; and
a surgical occlusion device configured to position the strip within the anatomical cavity and to support the biocompatible patch over an opening defined by the anatomical cavity while a user securely attaches the patch to the opening.

2. The system of claim 1, wherein the surgical procedure is an open heart surgery.

3. The system of claim 2, wherein the anatomical cavity is a left atrial appendage.

4. The method of claim 3, wherein the biocompatible patch and strip are bovine pericardium.

5. A method for occluding an anatomical cavity during a surgical procedure, comprising:
providing a biocompatible patch sized to cover the opening defined by the anatomical cavity and having a top side and a bottom side;
providing a biocompatible strip having a first end and a second end;
securing a first end of the strip to a central portion of the bottom side of the patch to define a plug assembly;
packing the strip into the anatomical cavity;
securing the patch to the opening defined by the anatomical cavity.

6. The method of claim 5, wherein securing a first end of the strip to a central portion of the bottom side of the patch to define a plug comprises suturing.

7. The method of claim 5, wherein securing the patch to the opening defined by the anatomical cavity comprises suturing.

8. The method of claim 5, wherein the surgical procedure is an open heart surgery.

9. The method of claim 8, wherein the anatomical cavity is a left atrial appendage.

10. The method of claim 5, wherein the biocompatible patch and strip are bovine pericardium.

11. The method of claim 10, further comprising scraping tissue comprising the anatomical cavity to promote adhesion of the bovine pericardium to the opening defined by the anatomical cavity.

12. The method of claim 5, further comprising:
reducing the vertical profile of the strip prior to packing it into the anatomical cavity;
providing a device for depositing at least the strip into an anatomical cavity;
inserting a terminal end of the device for depositing at least the strip, corresponding with the second end of the strip, into the anatomical cavity; and
removing the device for depositing at least the strip from the anatomical cavity.

13. A biocompatible plug assembly for occluding an anatomical cavity during surgery, comprising:
a biocompatible patch having a top side and a bottom side, the patch being sized and configured to cover an opening defined by the anatomical cavity, and
a biocompatible strip defining a flexible, elongated body foldable upon itself to reduce its vertical profile, the strip having a first end and a second end, the first end configured to be secured to a central portion of the bottom side of the patch.

14. The plug assembly of claim 13, wherein the biocompatible patch and strip comprise biocompatible bovine pericardium.

15. The plug assembly of claim 13, wherein, when secured to the opening defined by the anatomical cavity, the biocompatible plug assembly is operative to prevent fluid or material passage between the anatomical cavity and surrounding structures.

16. The plug assembly of claim 13, wherein the anatomical cavity is an atrial appendage, and wherein when secured to the opening defined by the atrial appendage, the biocompatible plug assembly is operative to prevent any passage of blood clots from the atrial appendage.
